(19) United States
(12) Patent Application Publication
(10) Pub. No.: US 2006/0148697 A1
(43) Pub. Date: Jul. 6, 2006

(54) Methods for treating and preventing brain cancers

(75) Inventors: Richard Lloyd Bowen, Raleigh, NC (US); Christopher W. Gregory, Cary, NC (US)

Correspondence Address: COVINGTON & BURLING;ATTN: PATENT DOCKETING, 1201 PENNSYLVANIA AVENUE, N.W., WASHINGTON, DC, 20004-2401, US

(73) Assignee: Voyager Pharmaceutical Corporation, Raleigh, NC (US)

(21) Appl. No.: 11/180,668

(22) Filed: Jul. 14, 2005

Related U.S. Application Data
(63) Continuation-in-part of application No. 10/321,579, filed on Dec. 18, 2002.
(60) Provisional application No. 60/340,502, filed on Dec. 19, 2001.
(60) Provisional application No. 60/385,577, filed on Jun. 5, 2002.
(60) Provisional application No. 60/369,857, filed on Apr. 5, 2002.
(60) Provisional application No. 60/385,576, filed on Jun. 5, 2002.
(60) Provisional application No. 60/383,624, filed on May 29, 2002.
(60) Provisional application No. 60/385,560, filed on Jun. 5, 2002.
(60) Provisional application No. 60/385,559, filed on Jun. 5, 2002.
(60) Provisional application No. 60/385,561, filed on Jun. 5, 2002.
(60) Provisional application No. 60/385,575, filed on Jun. 5, 2002.

(51) Int. Cl. A61K 38/22; A61K 38/09

(52) U.S. Cl. 514/12; 514/14; 514/15

(57) ABSTRACT

Methods are provided for treating brain cancer, preventing or slowing proliferation of cells of prostate origin, preventing brain cancer in a patient at risk of contracting brain cancer, preventing or inhibiting an upregulation of the cell cycle in brain-derived cells in a patient, and decreasing the level of brain cancer-specific antigen in a patient.

### RELATED APPLICATIONS

This application is a continuation-in-part of U.S. patent application Ser. No. 10/321,579, filed Dec. 18, 2002, which claims priority to U.S. Provisional Application No. 60/340,502, filed Dec. 19, 2001; U.S. Provisional Application No. 60/369,857, filed Apr. 5, 2002; U.S. Provisional Application No. 60/383,624, filed May 29, 2002; U.S. Provisional Application No. 60/385,577, filed Jun. 5, 2002; U.S. Provisional Application No. 60/385,576, filed Jun. 5, 2002; U.S. Provisional Application No. 60/385,560, filed Jun. 5, 2002; U.S. Provisional Application No. 60/385,559, filed Jun. 5, 2002; U.S. Provisional Application No. 60/385,561, filed Jun. 5, 2002; and U.S. Provisional Application No. 60/385,575, filed Jun. 5, 2002. The entirety of each of the above-identified applications is hereby incorporated by reference herein.

### FIELD OF THE INVENTION

The present invention relates to methods for treating, preventing, delaying, or mitigating brain cancer, for decreasing the level of brain cancer-specific markers, or for preventing or slowing proliferation of cells of brain origin.

### BACKGROUND

“Brain cancer” means any abnormally increased proliferation of any type of neuronal cells, or any other cancer that has metastasized into the central nervous system. Examples of brain cancers include, but are not limited to, neuroma, anaplastic astrocytoma, neuroblastoma, glioma, glioblastoma multiforme, astrocytoma, meningioma, pituitary adenoma, primary CNS lymphoma, medulloblastoma, ependymoma, sarcoma, oligodendroglioma, medulloblastoma, spinal cord tumor, and schwannoma. (Hill J R, Kuriyama N, Kuriyama H, Israel M A, Molecular genetics of brain tumors, Arch Neurol Apr;56(4):439-41 (1999)).

Most neuronal cells—that is, cells that comprise or are found in the central nervous system, including, for example, neurons, microglia, and astrocytes—are “terminally differentiated,” meaning that they no longer possess the ability to complete the cell cycle. (Jacobsen M, Histogenesis and morphogenesis of cortical structures, in Developmental Neurobiology, M. Jacobsen, ed., Plenum, New York, N.Y., 1991, pp. 401-451). Although terminally differentiated neuronal cells may be able to enter the cell cycle, they are unable to complete the process and usually undergo apoptosis (cell death). (Multani A S, Ozen M, Narayan S, Kumar V, Chandra J, McConkey D J, Newman R A, Pathak S, Caspase-dependent apoptosis induced by telomere cleavage and TRF2 loss, Neoplasia Jul-Aug;2(4):339-45 (2000)). Brain cancers may result when terminally differentiated neuronal cells lose the protective ability to apoptose and are able to complete the cell cycle, resulting in abnormally increased cell proliferation. (Hahn W C, Meyerson M, Telomerase activation, cellular immortalization and cancer, Ann Med Mar;33(2):123-9 (2001)).

Currently available treatments for brain cancers include surgery (tumor resection), radiation therapy, non-invasive high dose radiation with a “gamma knife,” chemotherapy, and combinations of the aforementioned treatments. It is estimated that in the year 2005, a total of 18,500 new brain cancers will be diagnosed and 12,760 deaths will be attributed to brain cancers in the United States. Accordingly, there is a need in the art for therapeutically effective treatments and preventative measures for brain cancers.

### SUMMARY

According to the present invention, an upregulation in the cell cycle, caused by increased mitogenic stimulus, contributes to the development of brain cancers by causing abnormally increased proliferation of neuronal cells that have lost the ability to apoptose.

In accordance with the present invention, and contrary to conventional teachings, an upregulation of the cell cycle associated with abnormally increased proliferation of neuronal cells is caused, at least in part, by an increase in blood level, production, function, or activity of luteinizing hormone (LH) or follicle stimulating hormone (FSH). Thus, the present invention encompasses preventing or treating brain cancer by administering an agent that decreases or regulates levels, production, function, or activity of LH or FSH.

The present invention provides that suppression of autocrine/paracrine gonadotropin releasing hormone (GnRH) signaling in the brain requires doses of GnRH agonists that are significantly higher than those required to suppress endocrine GnRH signaling at the level of the pituitary. The present invention further provides that hormones of the hypothalamic-pituitary-gonadal (HPG) axis function not only in an endocrine fashion to modulate brain cell function but also in an autocrine/paracrine fashion to regulate brain cell function.

A brief overview of the hypothalamic-pituitary-gonadal (HPG) hormonal axis is presented with reference to FIG. 11. In humans and many other mammals, the centrally produced hormones include gonadotropin releasing hormone (GnRH) from the hypothalamus; and gonadotropins, luteinizing hormone (LH), and follicle stimulating hormone (FSH) from the pituitary. Peripherally produced hormones include estrogen, progesterone, testosterone, and inhibins that are primarily of gonadal origin, while activins and follistatin are produced in all tissues including the gonads (Carr B R, in Williams Textbook of Endocrinology, J D Wilson, D W Foster, H M Kronenberg, and P R Larsen, eds. (Philadelphia Pa., WB Saunders Co.), pp. 751-817 (1998)).

The levels of each of these hormones are regulated by a complex feedback loop. Activins, which are produced by most tissues, stimulate gonadotropin releasing hormone (GnRH) secretion from the hypothalamus which stimulates the anterior pituitary to secrete the gonadotropins LH and FSH which in turn enter the blood stream and bind to receptors in the gonads and stimulate oogenesis/spermatogenesis as well as sex steroid and inhibin production. (Reichlin S. Neuroendocrinology; in Wilson J D, Foster D W, Kronenberg H M, Larsen P R 9eds): William's Textbook of Endocrinology, ed. 9. Philadelphia, Saunders, 1998, pp. 165-248). The sex steroids and inhibin then feedback to the hypothalamus and pituitary, resulting in a decrease in gonadotropin secretion. (Thorner M, Vance M, Laws E Jr., Horvath E, Kovacs K. The anterior pituitary; in Wilson J D, Foster D W, Kronenberg H M, Larsen P R 9eds): William's Textbook of Endocrinology, ed. 9. Philadelphia, Saunders, 1998, pp. 249-340).

Receptors for luteinizing hormone releasing hormone (LHRH) have been detected in meningiomata, glioblastoma multiforme, gliomata and chordoma using LHRH binding assays, demonstrating a possible autocrine signaling loop in brain cancers (van Groeninghen J C, Kiesel L, Winkler D, Zwirner M. Effects of luteinising-hormone-releasing hormone on nervous-system tumors. Lancet 352:372-373, 1998).

GnRH agonists are the most commonly used type of hormonal therapy for prostate cancer. These are analogues of the endogenous GnRH decapeptide with specific amino acid substitutions. Replacement of the GnRH carboxy-terminal glycinamide residue with an ethylamide group greatly increases the affinity these analogues possess for the GnRH receptor compared to the endogenous peptide. Many of these analogues also have a longer half-life than endogenous GnRH (Millar R P, Lu Z L, Pawson A J, Flanagan C A, Morgan K, Maudsley S R. Gonadotropin-releasing hormone receptors. Endocrine Reviews 25:235-275, 2004). Administration results in an initial increase in serum gonadotropin concentrations that persists for several days (there is also a corresponding increase in testosterone in men and estrogen in pre-menopausal women). This is followed by a precipitous decrease in gonadotropins. This suppression is due to the loss of GnRH signaling due to down regulation of pituitary GnRH receptors (Belchetz P E, Plant T M, Nakai Y, Keogh E J, Knobil E. Hypophysial responses to continuous and intermittent delivery of hypothalamic gonadotropin-releasing hormone. Science 202:631-633, 1978). This is thought to be secondary to the increased concentration of ligand, the increased affinity of the ligand for the receptor and the continuous receptor exposure to ligand as opposed to the intermittent exposure that occurs with physiological pulsatile secretion.

While customary doses of GnRH agonists and antagonists may generally be considered to be adequate to suppress endocrine influences of hormones of the HPG axis by lowering their serum concentrations, these same doses of GnRH antagonists and agonists are believed to be subtherapeutic when it comes to adequately suppressing local tissue production of these hormones.

The underlying rationale for treating brain cancers with hormonal therapy is that abnormal cell division in malignant brain tissues may be driven by elevated levels of gonadotropins. It is believed that, by reducing the level of gonadotropins in the serum and brain tissue of patients with brain cancers, brain cancer can be treated, prevented, delayed, or mitigated.

Among the goals of the present invention is treatment, mitigation, slowing the progression of, or preventing brain cancers by achieving higher tissue levels of GnRH agonists and/or GnRH antagonists, whether by administering more of such drugs, by preventing degradation of such drugs once administered, by delivering the drugs at a site where they are needed, by a combination of these methods, or by other methods.

The present invention relates to methods for treating, mitigating, slowing the progression of, or preventing brain cancer, or preventing or slowing proliferation of cells of brain origin, or for decreasing the level of a brain cancer-specific marker in a patient, by administering high doses of at least one physiological agent, such as a GNRH agonist or a GnRH antagonist, that decreases or regulates the blood or tissue levels, expression, production, function, or activity of LH, LH receptors, FSH, FSH receptors, androgenic steroids, androgenic steroid receptors, activins, or activin receptors, or administering a physiological agent that increases or regulates the blood or tissue levels, expression, production, function, or activity of GnRH, inhibins, beta-glycan, or follistatins.

The invention further encompasses, for example, a method of preventing or inhibiting an upregulation of the cell cycle in brain-derived cells by administering high doses of at least one physiological agent that is a GnRH agonist or antagonist, effective to reduce local tissue production of hormones of the hypothalamic-pituitary-gonadal (HPG) axis. In embodiments, the physiological agent is leuprolide, and the amount administered is in the range of approximately at least 15 mg/month. In other embodiments, the amount of leuprolide administered is in the range of at least about 20 mg/month, or at least 37.5 mg/month. In other embodiments, the physiological agent is an agent other than leuprolide, and the amount administered is an amount sufficient to produce the same or similar physiological effects as at least about 15 mg of leuprolide per month, or at least about 20 mg of leuprolide per month, or at least about 37.5 mg of leuprolide per month. In this specification, the term “physiologically equivalent dose” to a dose of a first physiological agent means a dose of a second physiological agent that achieves the same or similar physiological responses as the dose of the first physiological agent. The invention also encompasses, as another example, a method for treating brain cancer in a patient having brain cancer comprising administering to the patient a physiological agent that decreases the degradation of GnRH agonists or GnRH antagonists, increases the half-life of GnRH agonists or GnRH antagonists, or increases brain tissue levels of GnRH agonists or GnRH antagonists within the patient.

The invention also encompasses, as another example, a method for treating brain cancer in a patient having brain cancer comprising administering to the patient a physiological agent that decreases the degradation of GnRH agonists or GnRH antagonists, increases the half-life of GnRH agonists or GnRH antagonists, or increases brain tissue levels of GnRH agonists or GnRH antagonists within the patient.

Leuprolide acetate is an example of a GnRH agonist used in the treatment of a cancer, i.e., prostate cancer. Approved GnRH agonists and antagonists, dosage levels and plasma/serum levels of active medication are as follows (according to their approved labelling): LUPRON® DEPOT 3.75 mg 1 month injection gives a mean plasma leuprolide concentration of 4.6-10.2 ng/ml at 4 hours postdosing; LUPRON® DEPOT 7.5 mg 1 month injection gives a mean plasma leuprolide concentration of 20 ng/ml at 4 hours and 0.36 ng/ml at 4 weeks; LUPRON® DEPOT-PED 11.25 mg 1 month injection gives a mean plasma leuprolide concentration of 1.25 ng/ml at 4 weeks; LUPRON® DEPOT-PED 15 mg injection gives a mean plasma leuprolide concentration of 1.59 ng/ml at 4 weeks; LUPRON® DEPOT 22.5 mg 3 month injection gives a mean plasma leuprolide concentration of 48.9 ng/ml at 4 hours and 0.67 ng/ml at 12 weeks; LUPRON® DEPOT 30 mg 4 month injection gives a mean plasma leuprolide concentration of 59.3 ng/ml at 4 hours and 0.3 ng/ml at 16 weeks; VIADUR® 72 mg 12 month implantation gives a mean serum leuprolide concentration of 16.9 ng/ml at 4 hours and 2.4 ng/ml at 24 hours with a 0.9 ng/ml mean serum concentration for 12 months; ELIGARD® 7.5 mg 1 month injection gives a mean serum leuprolide concentration of 25.3 ng/ml at 5 hours and a serum level range of 0.28-2.0 ng/ml for one month; ZOLADEX® 3.6 mg 1 month (serum levels unavailable); ZOLADEX® 10.8 mg 3 month (serum levels unavailable); SYNAREL® 200 micrograms twice daily (serum levels unavailable); TRELSTAR DEPOT 3.75 mg 1 month gives a mean plasma triptorelin concentration of 28.43 ng/ml at 4 hours and declines to 0.084 ng/ml at 4 weeks; Supprelin 200 μg/ml, 500 μg/ml and 1000 μg/ml for daily injection (serum levels unavailable); SUPREFACT® 6.3 mg 2 month implant or 500 μg every 8 hours for 7 days followed by 200 μg per day (serum levels unavailable); CETROTIDE® 0.25 mg daily or 3.0 mg every 4 days gives a mean plasma cetrorelix concentration of 4.97 ng/ml or 28.5 ng/ml at 4 hours, respectively; PLENAXIS® 100 mg given on days 1, 15 and 28 and every 4 weeks afterward (serum levels unavailable); ANTAGON 250 μg daily gives a mean plasma ganirelix concentration of 14.8 ng/ml at 4 hours.

### BRIEF DESCRIPTION OF THE DRAWINGS

FIG. 1A presents results of an in vitro experiment in which leuprolide acetate was administered to cells of the LN229 adult glioblastoma brain cancer cell line on the initial day of a seven-day period.

FIG. 1B presents results of an in vitro experiment in which leuprolide acetate was administered to cells of the LN229 adult glioblastoma brain cancer cell line on each day of a five-day period.

FIG. 1C presents results of an in vitro experiment in which leuprolide acetate was administered to cells of the LN229 adult glioblastoma brain cancer cell line on each day of a seven-day period.

FIG. 2A presents results of an in vitro experiment in which leuprolide acetate was administered to cells of the U87-MG adult astrocytoma brain cancer cell line on each day of a seven-day period.

FIG. 2B presents results of an in vitro experiment in which leuprolide acetate was administered to cells of the U87-MG adult astrocytoma brain cancer cell line on each day of a seven-day period.

FIG. 2C presents results of an in vitro experiment in which leuprolide acetate was administered to cells of the U87-MG adult astrocytoma brain cancer cell line twice per day of a five-day period.

FIG. 3 presents results of two in vitro experiments (averaged) in which leuprolide acetate was administered to cells of the U118-MG adult glioblastoma brain cancer cell line twice daily for five days.

FIG. 4A presents results of an in vitro experiment in which leuprolide acetate was administered to cells of the DAOY pediatric medulloblastoma brain cancer cell line on the first day of a seven-day period.

FIG. 4B presents results of an in vitro experiment in which leuprolide acetate was administered to cells of the DAOY pediatric medulloblastoma brain cancer cell line on each day of a six-day period.

FIG. 4C presents results of an in vitro experiment in which leuprolide acetate was administered to cells of the DAOY pediatric medulloblastoma brain cancer cell line on each day of a seven-day period.

FIG. 5A presents results of an in vitro experiment in which leuprolide acetate was administered to cells of the SK-N-MC pediatric neuroblastoma brain cancer cell line on the first day of a seven-day period.

FIG. 5B presents results of an in vitro experiment in which leuprolide acetate was administered to cells of the SK-N-MC pediatric neuroblastoma brain cancer cell line on each day of a six-day period.

FIG. 5C presents results of a replicate in vitro experiment in which leuprolide acetate was administered to cells of the SK-N-MC pediatric neuroblastoma brain cancer cell line on each day of a seven-day period.

FIG. 6A presents tumor growth data from an experiment in which human LN229 brain cancer cells were injected as xenografts into nude mice that were one week prior treated with placebo or leuprolide implants.

FIG. 6B presents tumor growth rates from tumors represented in FIG. 6A.

FIG. 7A presents tumor growth data from an experiment in which human U87-MG brain cancer cells were injected as xenografts into nude mice that were concurrently implanted with placebo or leuprolide implants.

FIG. 7B presents tumor growth rates from tumors represented in FIG. 7A.

FIG. 8A presents tumor growth data for small tumors (≦4×V₀) from an experiment in which human U87-MG brain cancer cells were injected as xenografts into nude mice that were one week prior implanted with placebo or leuprolide implants.

FIG. 8B presents tumor growth rates from the small tumors represented in FIG. 8A.

FIG. 8C presents tumor growth data for large tumors (≧24×V₀) from an experiment in which human U87-MG brain cancer cells were injected as xenografts into nude mice that were one week prior implanted with placebo or leuprolide implants.

FIG. 8D presents tumor growth rates from the large tumors represented in FIG. 8C.

FIG. 9A presents tumor growth data from an experiment in which human DAOY brain cancer cells were injected as xenografts into nude mice that were subsequently dosed with placebo or leuprolide implants one week after injection.

FIG. 9B presents tumor growth rates from tumors represented in FIG. 9A.

FIG. 10A presents tumor growth data for small tumors from an experiment in which SK-N-MC brain cancer cells were injected as. xenografts into nude mice that were subsequently dosed with placebo or leuprolide implants one week after injection.

FIG. 10B presents tumor growth rates from tumors represented in FIG. 10A.

FIG. 10C presents tumor growth data for large tumors from an experiment in which SK-N-MC brain cancer cells were injected as xenografts into nude mice that were subsequently dosed with placebo or leuprolide implants one week after injection.

FIG. 10D presents tumor growth rates from tumors represented in FIG. 10C.

FIG. 11 is a schematic overview of the hypothalamic-pituitary-gonadal hormonal axis.

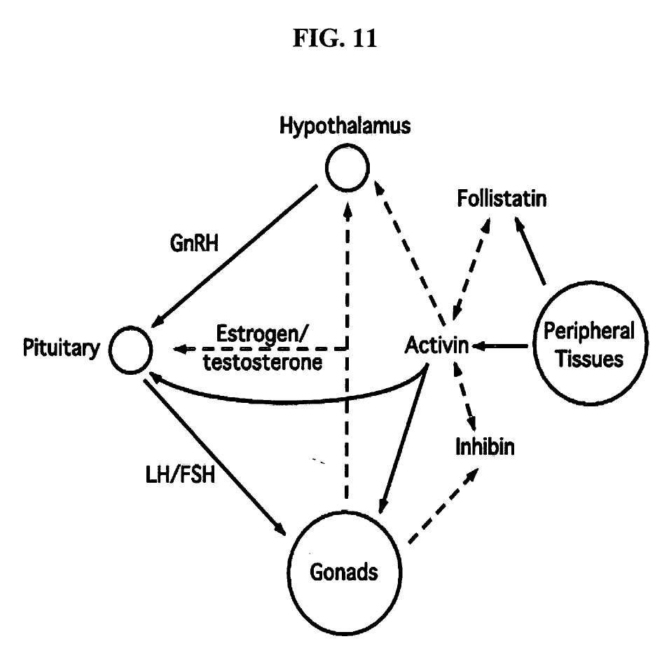

### DETAILED DESCRIPTION

The present invention encompasses methods of preventing or treating brain cancer, or preventing or slowing proliferation of neuronal cells, or inhibiting or preventing upregulation of the cell cycle by administering an agent that decreases or regulates blood and brain levels, production, function, or activity of LH or FSH (an “LH/FSH-inhibiting agent”). According to the invention, the LH/FSH-inhibiting agent comprises one or more of gonadotropin releasing hormone (GnRH); leuprolide; triptorelin; buserelin; nafarelin; desorelin; histrelin; goserelin; follistatin; a compound that stimulates the production of follistatin; a GnRH antagonist; a GnRH receptor blocker; citrorelix; abarelix; a vaccine or antibody that stimulates the production of antibodies that inhibit the activity of any of LH, FSH, or GnRH; a vaccine or antibody that stimulates the production of antibodies that block an LH receptor, an FSH receptor, or a GnRH receptor; a compound that regulates expression of an LH or FSH receptor; a compound that regulates post-receptor signaling of an LH or FSH receptor; or a physiologically acceptable analog, metabolite, precursor, or salt of any of the foregoing LH/FSH-inhibiting agents.

In embodiments of the invention, the blood level, production, function, or activity of LH or FSH is decreased or regulated to be near a target blood level, a target production, a target function, or a target activity of LH or FSH, respectively, occurring at or near the time of greatest reproductive function, which in humans corresponds to 18 to 35 years of age.

In other embodiments of the invention, the blood level, production, function, or activity of LH or FSH is decreased or regulated to be approximately as low as possible without unacceptable adverse side effects. An unacceptable adverse side effect is an adverse side effect that, in the reasonable judgment of one of ordinary skill in the art, has costs that outweigh the benefits of treatment.

In yet other embodiments, the blood level, production, function, or activity of LH or FSH is decreased or regulated to be undetectable or nearly undetectable by conventional means known in the art, meaning less than 0.7 mIU/mL for both LH and FSH in a clinical laboratory, and lower in a commercial laboratory.

Embodiments of the present invention include administration of one or more LH/FSH-inhibiting agents that can be used to decrease or regulate the blood level, production, function, or activity of LH or FSH. In certain embodiments of the invention, GnRH or a GnRH analog can be administered to decrease or regulate the brain or blood level, production, function, or activity of LH or FSH. Studies have shown that increased levels of GnRH or its analogs will result in significant decreases in LH and FSH levels. (Thomer M O, et al., The anterior pituitary, in Williams Textbook of Endocrinology 9^{th} edition, eds. Wilson J D, Foster D W, Kronenberg H, Larsen P R, 269, W. B. Saunders Company, Philadelphia, Pa. (1998)). For example, leuprolide, a GnRH analog, has been shown to increase pituitary secretion of LH and FSH for several days after initial administration. (Mazzei T, et al., Pharmacokinetics, endocrine and antitumor effects of leuprolide depot (TAP-144-SR) in Advanced Prostatic Cancer: A Dose Response Evaluation, Drugs in Experimental and Clinical Research, 15:373-387 (1989)). Thereafter, pituitary GnRH receptors are down-regulated, resulting in a significant decrease in LH and FSH secretion. (Mazzei T, et al., Human pharmacokinetic and pharmacodynamic profiles of leuprorelin acetate depot in prostatic cancer patients, Journal of Internal Medicine Research, 18(suppl):42-56 (1 990)).

Examples of GnRH analogs that are useful in the present invention include leuprolide, triptorelin, buserelin, nafarelin, desorelin, histrelin, and goserelin. Other embodiments of LH/FSH-inhibiting agents include GnRH antagonists, GnRH receptor blockers, such as citrorelix and abberelix, and LH or FSH receptor blockers. Currently approved GnRH agonists and antagonists, dosage levels, and plasma/serum levels of active medication (according to package inserts and prescribing information) are as follows: LUPRON® DEPOT 3.75 mg 1 month injection gives a mean plasma leuprolide concentration of 4.6-10.2 ng/ml at 4 hours postdosing; LUPRON® DEPOT 7.5 mg 1 month injection gives a mean plasma leuprolide concentration of 20 ng/ml at 4 hours and 0.36 ng/ml at 4 weeks; LUPRON®0 DEPOT-PED 11.25 mg 1 month injection gives a mean plasma leuprolide concentration of 1.25 ng/ml at 4 weeks; LUPRON® DEPOT-PED 15 mg injection gives a mean plasma leuprolide concentration of 1.59 ng/ml at 4 weeks; LUPRON® DEPOT 22.5 mg 3 month injection gives a mean plasma leuprolide concentration of 48.9 ng/ml at 4 hours and 0.67 ng/ml at 12 weeks; LUPRON® DEPOT 30 mg 4 month injection gives a mean plasma leuprolide concentration of 59.3 ng/ml at 4 hours and 0.3 ng/ml at 16 weeks; VIADUR® 72 mg 12 month implantation gives a mean serum leuprolide concentration of 16.9 ng/ml at 4 hours and 2.4 ng/ml at 24 hours with a 0.9 ng/ml mean serum concentration for 12 months; ELIGARD® 7.5 mg 1 month injection gives a mean serum leuprolide concentration of 25.3 ng/ml at 5 hours and a serum level range of 0.28-2.0 ng/ml for one month; ZOLADEX® 3.6 mg 1 month (serum levels unavailable); ZOLADEX® 10.8 mg 3 month (serum levels unavailable); SYNAREL® 200 micrograms twice daily (serum levels unavailable); TRELSTAR DEPOT 3.75 mg 1 month gives a mean plasma triptorelin concentration of 28.43 ng/ml at 4 hours and declines to 0.084 ng/ml at 4 weeks; Supprelin 200 μg/ml, 500 μg/ml and 1000 μg/ml for daily injection (serum levels unavailable); SUPREFACT® 6.3 mg 2 month implant or 500 μg every 8 hours for 7 days followed by 200 μg per day (serum levels unavailable); CETROTIDE® 0.25 mg daily or 3.0 mg every 4 days gives a mean plasma cetrorelix concentration of 4.97 ng/ml or 28.5 ng/ml at 4 hours, respectively; PLENAXIS® 100 mg given on days 1, 15, and 28 and every 4 weeks afterward (serum levels unavailable); ANTAGON 250 μg daily gives a mean plasma ganirelix concentration of 14.8 ng/ml at 4 hours.

In still other embodiments of LH/FSH-inhibiting agents, vaccines or antibodies can be employed to stimulate the production of antibodies that recognize, bind to, block or substantially reduce the activity of LH, FSH, or GnRH. In other embodiments, vaccines or antibodies can be employed to stimulate the production of antibodies that recognize, bind to, or block the receptors for one of LH, FSH, or GnRH. Examples of such vaccines include the Talwar vaccine and the vaccine marketed under the trade name GONADIMMUNE® by Aphton Corporation. Other LH/FSH-inhibiting agents include compounds that regulate expression of LH and FSH receptors and agents that regulate post-receptor signaling of LH and FSH receptors.

In other embodiments of the invention, a sex steroid hormone, such as estrogen, progesterone, or testosterone, or an analog thereof, may be co-administered with an LH/FSH-inhibiting agent. Through a negative feedback loop, the presence of estrogen, progesterone, or testosterone signals the hypothalamus to decrease the secretion of GnRH. (Gharib S D, et al., Molecular biology of the pituitary gonadotropins, Endocrine Reviews, 11:177-199 (1990); Steiner R A, et al., Regulation of luteinizing hormone pulse frequency and amplitude by testosterone in the adult male rat, Endocrinology, 111:2055-2061 (1982)). The subsequent decrease in GnRH decreases the secretion of LH and FSH. (Thomer M O, et al., The anterior pituitary, in Williams Textbook of Endocrinology, 9th edition, eds. Wilson J D, Foster D W, Kronenberg H, Larsen P R, 269, W.B. Saunders Company, Philadelphia, Pa. (1998)). Thus, according to the present invention, co-administration of estrogen, progesterone, or testosterone further decreases secretion of LH or FSH, and thereby inhibits upregulation of the cell cycle, sometimes with synergistic effects. Moreover, because administration of the LH/FSH-inhibiting agents described above may have the undesired side-effect of reducing the natural production of sex steroids, the present invention also encompasses co-administration of sex steroids in order to replenish the sex steroids.

Since these GnRH agonists are peptides, they are generally not amenable to oral administration. Therefore, they are usually administered subcutaneously, intra-muscularly, or via nasal spray. GnRH agonists are highly potent with serum concentrations of less than 1 ng/ml of leuprolide acetate required for testosterone suppression (Fowler, J. E., Flanagan, M., Gleason, D. M., Klimberg, I. W., Gottesman, J. E., and Sharifi, R. (2000) Evaluation of an implant that delivers leuprolide for 1 year for the palliative treatment of prostate cancer. Urol. 55:639-642). Due to their small size and high potency, GnRH agonists are also often considered to be ideal for use in long-acting depot delivery systems. At least ten such products are currently marketed in the United States. The duration of action of these products ranges from one month to one year. Leuprolide acetate has been on the market for close to two decades and continues to demonstrate a favorable side effect profile. Most of the side effects such as hot flashes and osteoporosis can be attributed to the loss of sex steroid production (Stege, R. (2000). Potential side-effects of endocrine treatment of long duration in prostate cancer. Prostate Suppl. 10:38-42).

Experimental Design

Cell growth assays were performed using two different methodologies as described below. LN229 (ATCC CRL-2611) cells were prepared by plating in Dulbecco's modified Eagle's medium with 4 mM L-glutamine adjusted to contain 1.5 g/L sodium bicarbonate and 4.5 g/L glucose, 95%; fetal bovine serum, 5%. U87-MG (ATCC HTB-14) cells were prepared by plating in Minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate, 90%; fetal bovine serum, 10%. U118-MG (ATCC HTB-15) cells were prepared by plating in Dulbecco's modified Eagle's medium with 4 mM L-glutamine adjusted to contain 1.5 g/L sodium bicarbonate and 4.5 g/L glucose, 90%; fetal bovine serum, 10%. DAOY (ATCC HTB-186) cells were prepared by plating in Minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate, 90%; fetal bovine serum, 10%. SK-N-MC (ATCC HTB-10) cells were prepared by plating in Minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate, 90%; fetal bovine serum, 10%. For cell growth assays performed in a 96-well format, 5000 cells were plated per well and allowed to grow for 2 days prior to commencement of treatment. For assays performed in 60 mm×15 mm dishes, different numbers of cells were plated, depending on the cell line (2×10⁴ for LN229, DAOY and SK-N-MC; 5×10⁴ for U87-MG and U118-MG). After the cells were established, they were then counted in order to obtain a baseline before the various concentrations of leuprolide were administered. A 10 mM (12.25 mg/ml) solution of leuprolide acetate salt in phosphate buffered saline was prepared and diluted appropriately to obtain the desired final concentrations. Treatment concentrations were 0 M (control), 10⁻¹¹ M (shown as 1.00E-11, 0.012 ng/ml), 10⁻⁹ M (shown as 1.00E-9, 0.0012 μg/ml), 10⁻⁸ M (shown as 1.00 E-8, 0012 μg/ml), 10⁻⁷M (shown as 1.00E-7, 0.12 μg/ml) and 10⁻⁵ M (shown as 1.00E-5, 12.25 μg/ml). For 96-well format assays, the number of cells in each group was measured by incubating cells with WST-8 (2-(2-methoxy-4-nitrophenyl)-3-(4-nitrophenyl)-5-(2,4-disulfophenyl)-2H-tetrazolium, monosodium salt) which produces a water soluble formazan dye that was detected by measuring optical density (at 450 nm) using a μQuant™ Universal Microplate Spectrophotometer (Bio-Tek® Instruments, Inc., Winooski, Vt.). For the 60 mm dish assays, cells were counted by a blinded observer using a hemacytometer and a microscope. All treatment groups were performed in triplicate and the optical densities or cell numbers are presented as mean i standard deviation.

For brain cancer tumor xenograft studies, male nude:nude athymic mice from Harlan Sprague Dawley (Indianapolis, Ind.) were used. Mice were anesthetized with Domitor/Ketaset and placed under a warming lamp. Tumor cells were injected in Matrigel (BD Biosciences, Bedford, Md.) and implants were placed subcutaneously into anesthetized mice. Tumor measurements were carried out twice weekly using calipers, and length (l) and width (w) were converted to tumor volumes using the following equation: (w²×l)/2. All tumors within one treatment group were used to calculate average tumor volumes +standard deviations. To calculate tumor growth rates, tumor volumes were normalized to the initial tumor volume (V₀). When a single tumor was detectable in a treatment group, that tumor volume was used as V₀ for that treatment group and all tumors measured in that group that formed over time were used to calculate a growth rate (V/V₀). At the end of the experiments, mice were sacrificed by cervical dislocation, and tissues and blood were collected.

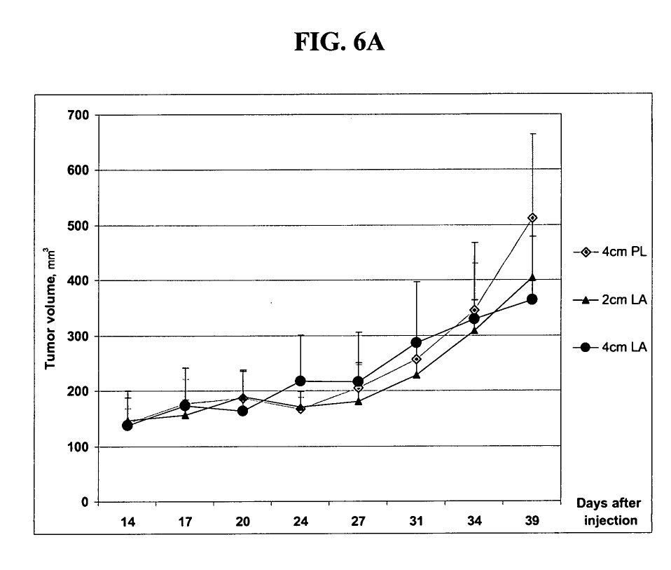

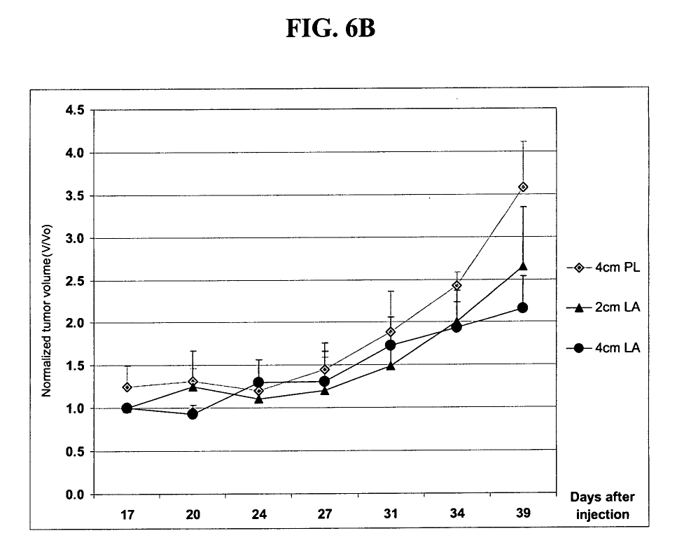

The DURIN-Leuprolide 2-month implant, available from Durect Corporation (Cupertino, Calif.), is a solid formulation comprising approximately 25-30 weight % leuprolide acetate dispersed in a matrix of poly (DL-lactide-co-glycolide). The implant is a cylindrical, opaque rod with nominal dimensions of 1.5 mm (diameter)×2.0 cm (length). The formulation provides 11.25 mg of leuprolide acetate per 2 cm rod, with a substantially uniform release profile. For tumor xenograft studies, the following doses were used: placebo (2 cm of formulation, 0 mg leuprolide acetate); low dose (2 cm of formulation, 11.25 mg leuprolide acetate); medium dose (3 cm of formulation; 16.875 mg leuprolide acetate); high dose (4 cm of formulation; 22.5 mg leuprolide acetate). Accordingly, in FIGS. 6A through 10D, the dimension on the right-hand axis refers to the length of these implants used for the particular experimental group, and the designations “LA” and “PL” refer respectively to leuprolide acetate and placebo.

Experiment 1

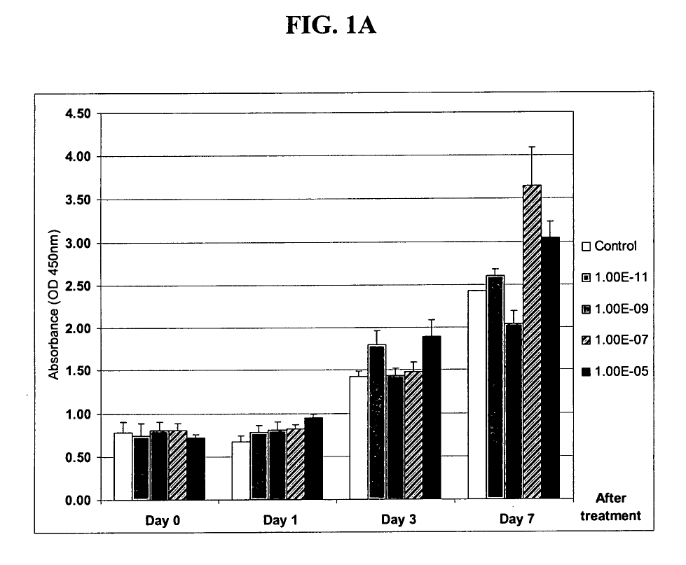

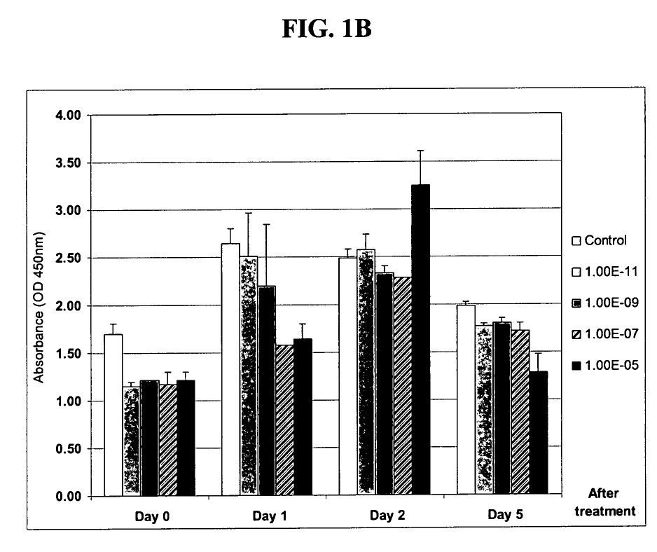

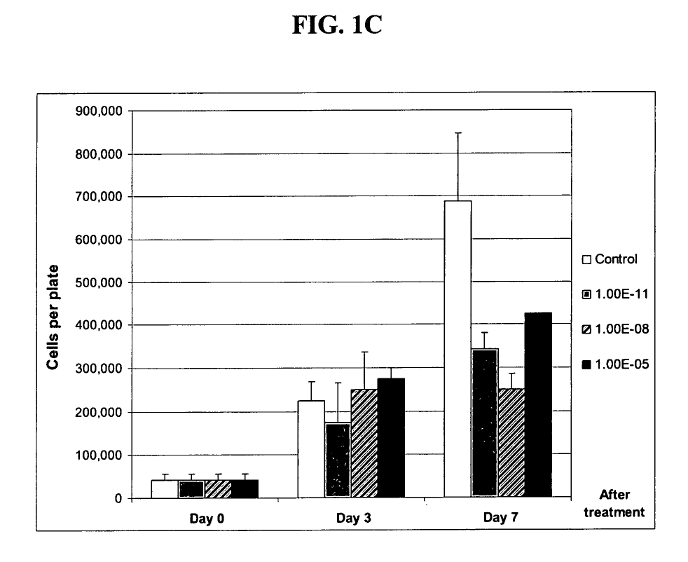

FIGS. 1A-C present results of a series of three experiments on the effects of administration of leuprolide acetate at various molar concentrations on the growth in number of cells of the LN229 adult glioblastoma brain cancer cell line. The source of this cell line was from a glioblastoma in the right frontal parieto-occipital cortex of a 60-year-old female patient.

In Experiment 1-A, each of five groups of cells from the LN229 cell line was prepared as described above and respectively treated with final concentrations of OM (phosphate buffered saline control), 10⁻¹¹M , 10⁻⁹M, 10⁻⁷M, or 10⁻⁵M leuprolide acetate solution at experiment commencement. The number of cells in each group was measured by incubating cells with WST-8 (as described in Experimental Design) at day 0 (experiment commencement) and on the first, third, and seventh days following commencement. FIG. 1A presents the results of this experiment. For each concentration of leuprolide acetate used in this experiment, and for each day on which absorbance was measured, FIG. 1A shows, on the vertical axis, the absorbance (450 nm), which indicates cell number as a function of optical density of the formazan dye product.

In Experiment 1-B, each of five groups of cells from the LN229 cell line was prepared as described above and respectively treated with final concentrations of OM (phosphate buffered saline control), 10⁻¹¹M , 10⁻⁹M, 10⁻⁷M, or 10⁻⁵M leuprolide acetate solution at experiment commencement and every day after commencement out to five days. The number of cells in each group was measured by incubating cells with WST-8 (as described in Experimental Design) at day 0 (experiment commencement) and on the first, second, and fifth days following commencement. FIG. 1B presents the results of this experiment. For each concentration of leuprolide acetate used in this experiment, and for each day on which absorbance was measured, FIG. 1B shows, on the vertical axis, the absorbance (450 nm), which indicates cell number as a function of optical density of the formazan dye product.

In Experiment 1-C, each of four groups of cells from the LN229 cell line was prepared as described above and respectively treated with final concentrations of OM (culture medium control), 10⁻¹¹M, 10⁻⁸M, or 10⁻⁵M leuprolide acetate solution at experiment commencement and every day after commencement up to seven days. The number of cells was measured by counting at experiment commencement and on the third and seventh days following commencement, using a hemacytometer and microscope by a blinded observer. FIG. 1C presents the results of this experiment. For each concentration of leuprolide acetate used in this experiment, and for each day on which cells were counted, FIG. 1C shows, on the vertical axis, the number of cells per plate.

As presented in FIGS. 1A-C, with daily administration of the highest concentration (10⁻⁵ M) of leuprolide acetate, growth of LN229 brain cancer cells was inhibited by approximately 40% compared to control cells (growing in culture medium which included no leuprolide acetate).

Experiment 2

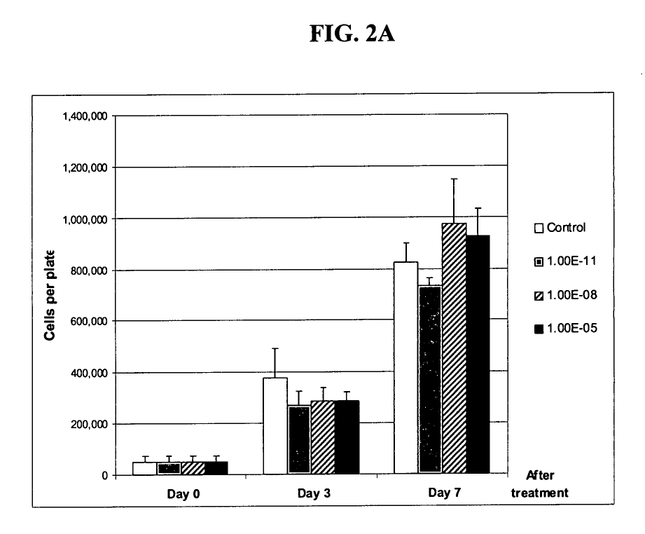

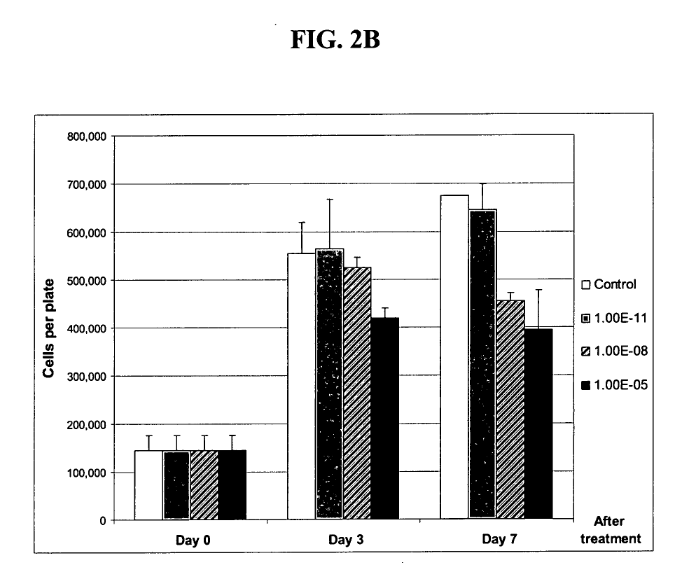

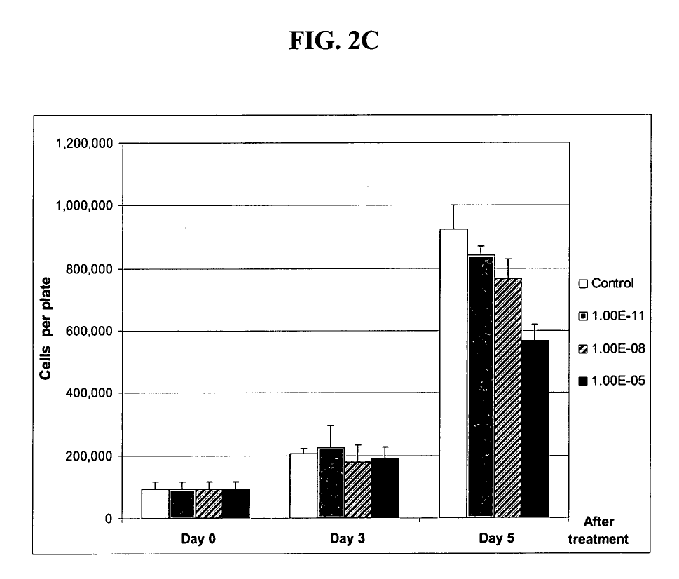

FIGS. 2A-C present results of a series of three experiments on the effects of administration of leuprolide acetate at various molar concentrations on the growth of cells of the U87-MG adult astrocytoma brain cancer cell line. The source of this cell line was from the brain of an adult female with a malignant glioma (astrocytoma).

FIG. 2A presents the results of the cell growth experiment with groups of U87-MG cells respectively administered the concentrations of leuprolide acetate identified in FIG. 2A at experiment commencement and on each day after commencement (0 M, 10⁻¹¹ M, 10⁻⁸ M, 10⁻⁵ M).

FIG. 2B presents the results of the cell growth experiment with groups of U87-MG cells respectively administered the concentrations of leuprolide acetate identified in FIG. 2B at experiment commencement and on each day after commencement (0 M, 10⁻¹¹ M, 10⁻⁸ M, 10⁻⁵ M).

FIG. 2C presents the results of the cell growth experiment with groups of U87-MG cells respectively administered the concentrations of leuprolide acetate identified in FIG. 2C at experiment commencement and twice per day after commencement (0 M, 10⁻¹¹ M, 10⁻⁸ M, 10⁻⁵ M).

As presented in FIGS. 2A-C, administration of the highest concentration (10⁻⁵M) of leuprolide acetate once or twice each day inhibited growth of U87-MG brain cancer cells by 40% compared to control cells growing in culture medium without leuprolide.

Experiment 3

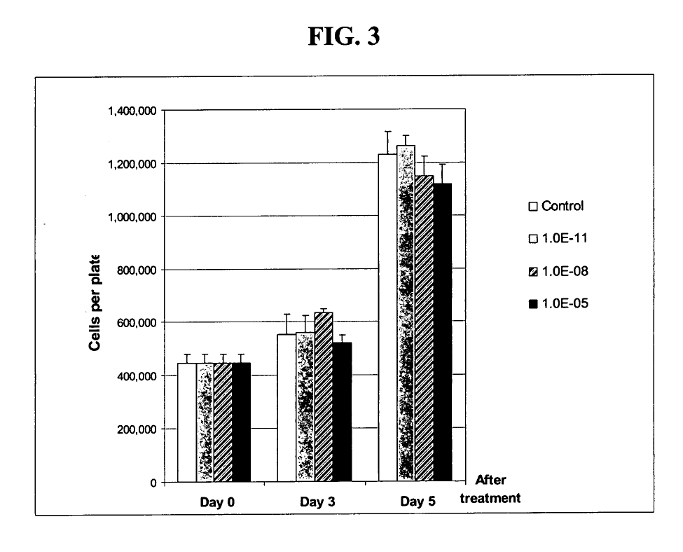

FIG. 3 presents results of two experiments on the effects of administration of leuprolide acetate at various molar concentrations on the growth of cells of the U118-MG adult glioblastoma cell line. The source of this cell line was from a stage III glioblastoma (astrocytoma) from the brain of a 50-year-old male patient.

FIG. 3 presents the results of two cell growth experiments (averaged) with groups of U118-MG cells respectively administered the concentrations of leuprolide acetate identified in FIG. 3 at experiment commencement and twice per day after commencement (0 M, 10⁻¹¹ M, 10⁻⁸ M, 10⁻⁵ M).

As presented in FIG. 3, administration of the highest concentration (10⁻⁵M) of leuprolide acetate twice each day inhibited growth of U118-MG brain cancer cells by 10% compared to control cells growing in culture medium without leuprolide.

Experiment 4

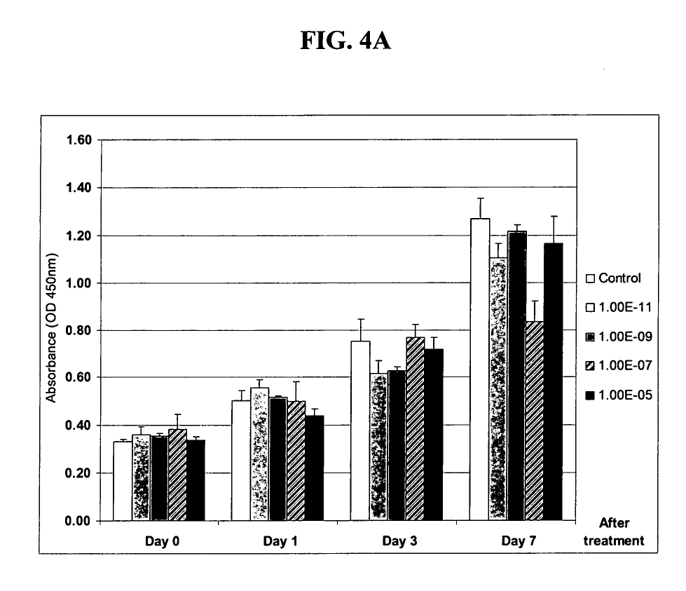

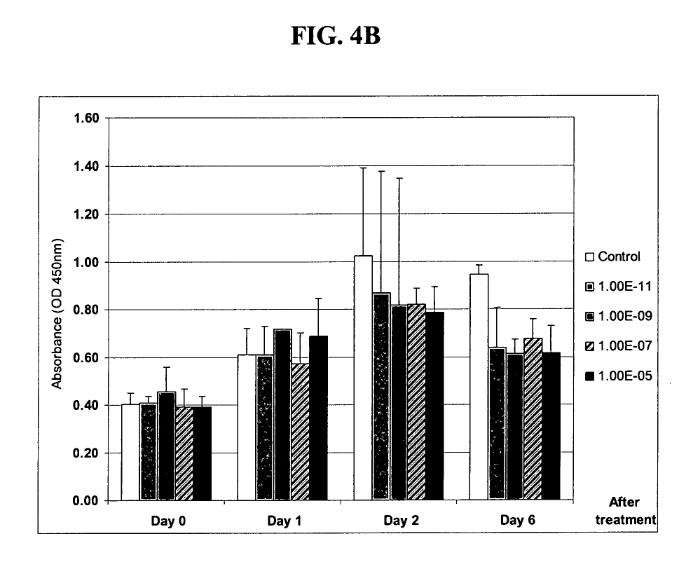

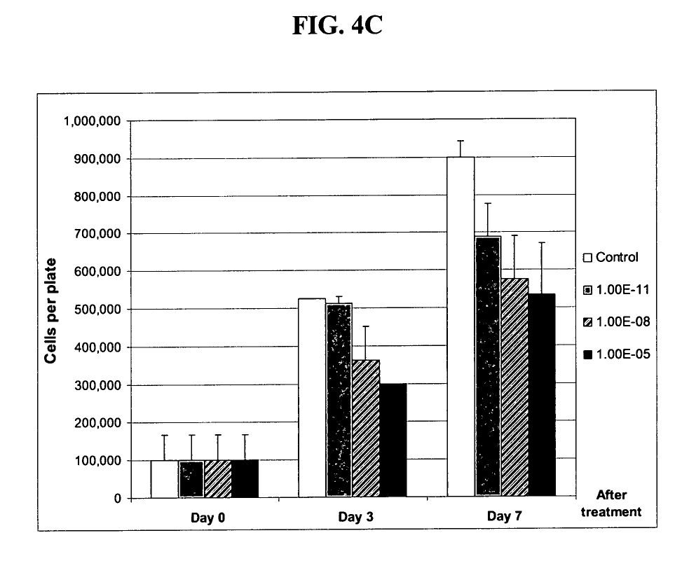

FIGS. 4A-C present results of a series of three experiments on the effects of administration of leuprolide acetate at various molar concentrations on the growth of cells of the DAOY pediatric medulloblastoma brain cancer cell line. The source of this cell line was from the brain of a 4-year-old male with a desmoplastic cerebellar medulloblastoma.

FIG. 4A presents the results of the cell growth experiment with groups of DAOY cells respectively administered the concentrations of leuprolide acetate identified in FIG. 4A at experiment commencement (0 M, 10⁻¹¹ M, 10⁻⁹ M, 10⁻⁷ M, and 10⁻⁵ M).

FIG. 4B presents the results of the cell growth experiment with groups of DAOY cells respectively administered the concentrations of leuprolide acetate identified in FIG. 4B at experiment commencement and on each day after commencement (0 M, 10⁻¹¹M, 10⁻⁹ M, 10⁻⁷ M, and 10⁻⁵ M).

FIG. 4C presents the results of a replicate cell growth experiment with groups of DAOY cells respectively administered the concentrations of leuprolide acetate identified in FIG. 4C at experiment commencement and on each day after commencement (0 M, 10⁻¹¹ M, 10⁻⁸ M, 10⁻⁵ M).

As presented in FIGS. 4B and 4C, administration of the highest concentration (10⁻⁵M) of leuprolide acetate once each day inhibited growth of DAOY brain cancer cells by 40% compared to control cells growing in culture medium without leuprolide.

Experiment 5

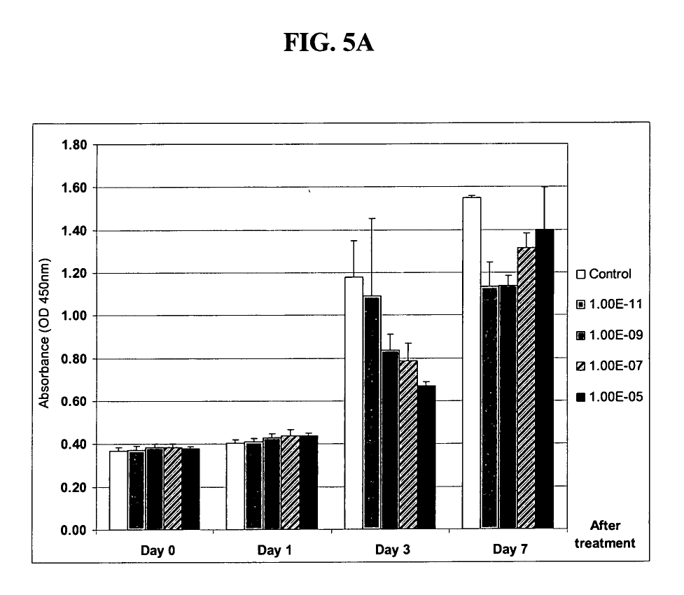

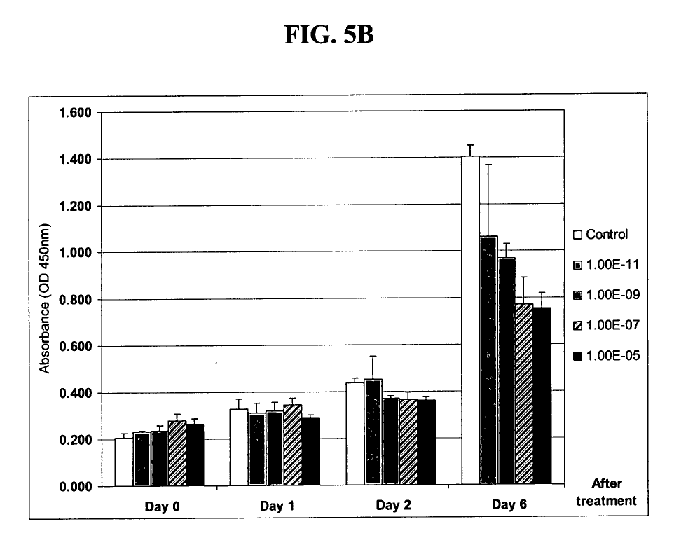

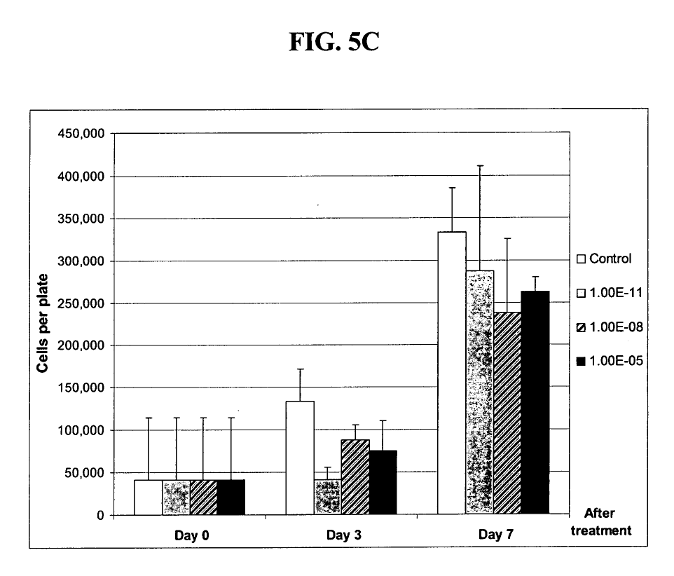

FIGS. 5A-C present the results of a series of three experiments on the effects of administration of leuprolide acetate at various molar concentrations on the growth of cells of the SK-N-MC brain cancer cell line. The source of this cell line is from a neuroepithelioma from the supra-orbital area of a 14-year-old female.

FIG. 5A presents the results of the cell growth experiment with groups of SK-N-MC cells respectively administered the concentrations of leuprolide acetate identified in FIG. 5A at experiment commencement (0 M, 10⁻¹¹ M, 10⁻⁹ M, 10⁻⁷ M, and 10⁻⁵ M).

FIG. 5B presents the results of the cell growth experiment with groups of SK-N-MC cells respectively administered the concentrations of leuprolide acetate identified in FIG. 5A at experiment commencement and each day after commencement (0 M, 10⁻¹¹ M, 10⁻⁹ M, 10⁻⁷ M, and 10⁻⁵ M).

FIG. 5C presents the results of the cell growth experiment with groups of SK-N-MC cells respectively administered the concentrations of leuprolide acetate identified in FIG. 5C at experiment commencement (0 M, 10⁻¹¹ M, 10⁻⁸ M, 10⁻⁵ M).

As presented in FIGS. 5B-C, administration of the highest concentration (10⁻⁵M) of leuprolide acetate once each day inhibited growth of SK-N-MC brain cancer cells by 30% compared to control cells growing in culture medium without leuprolide.

Experiment 6

FIGS. 6A and 6B present results of experiments in which 5×10⁶ cells of the LN229 human glioblastoma brain cancer cell line were injected bilaterally into three groups (two treatment groups and a control group), each with three mice. One week prior to the injection, a controlled-release leuprolide acetate formulation was implanted into each mouse from one of the groups. Two centimeters of leuprolide rod, providing 11.25 mg of leuprolide, or four centimeters of leuprolide rod, providing 22.5 mg of leuprolide, were implanted in each mouse of the treatment groups. Four centimeters of placebo rod (without leuprolide) were implanted one week prior to injection into each mouse of the control group.

FIG. 6A presents results of tumor xenograft growth over time in a placebo group and two leuprolide implant groups. As FIG. 6A shows, tumor volume measurements were commenced on the fourteenth day following injection, when tumors were detectable in all groups. By the thirty-ninth day following injection, tumors in the control group (n=6) had grown to approximately 510 mm³ on average, while tumors in the 2 cm treatment group (n=5) had grown to approximately 400 mm³ on average, and tumors in the 4 cm treatment group (n=6) had grown to 350 mm3 on average.

FIG. 6B presents results of measurement of tumor growth rate in each of the treatment and control groups for this experiment. As indicated with respect to FIG. 6A, on the fourteenth day following injection, tumors were first observed in all groups and these tumor sizes were used as V₀ for a calculation of growth rate (see Experimental Design). Tumor growth rates were similar in both groups until day 31 after injection, at which time tumors in the placebo group began to grow at a more rapid rate compared to tumors in leuprolide-treated mice.

As presented in FIGS. 6A and 6B, on day 39 after cell injection of LN229 brain cancer cells, tumors growing in mice treated with leuprolide were smaller by 25% compared to tumors growing in placebo mice, and the tumors in mice treated with 4 cm of leuprolide had doubled in size while tumors growing in placebo mice had increased almost three times over the initial tumor size (V₀).

Experiment 7

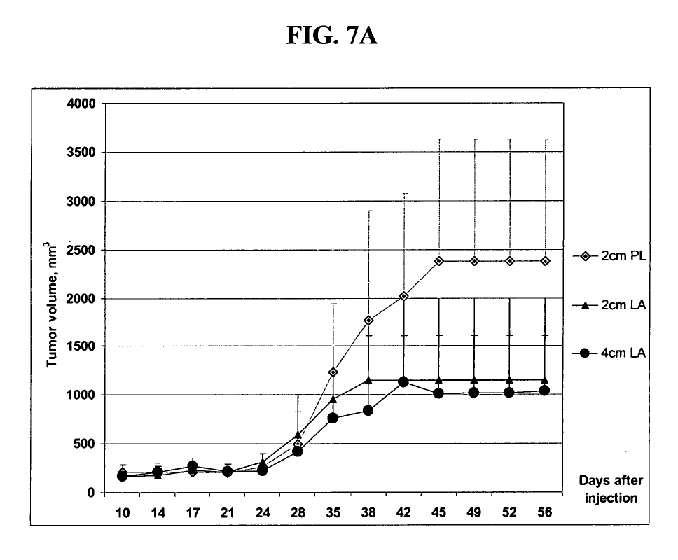

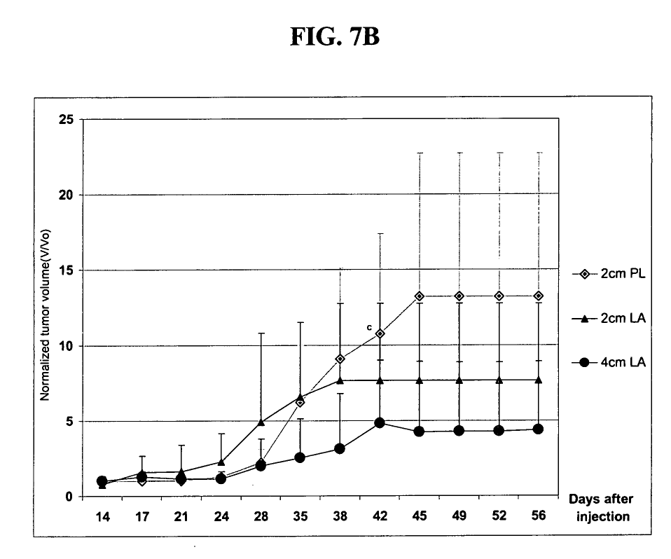

FIGS. 7A and 7B present results of experiments in which 5×10⁶ cells of the U87-MG human glioblastoma brain cancer cell line were injected bilaterally into three groups (two treatment groups and a control group), each with three mice. Concurrently with the cell injection, a controlled-release leuprolide acetate formulation was implanted into each mouse from one of the groups. Two centimeters of leuprolide rod, providing 11.25 mg of leuprolide, or four centimeters of leuprolide rod, providing 22.5 mg of leuprolide, were implanted in each mouse of the treatment groups. Four centimeters of placebo rod (without leuprolide) were implanted one week prior to injection into each mouse of the control group.

FIG. 7A presents results of tumor xenograft growth over time in the placebo group and the two leuprolide implant groups. As FIG. 7A shows, tumor volume measurements were commenced on the tenth day following injection, when tumors were detectable in all groups. By the forty-fifth day following injection, tumors in the control group (n=6) had grown to approximately 2400 mm³ on average, while tumors in the 2 cm treatment group (n=6) had grown to approximately 1100 mm³ on average, and tumors in the 4 cm treatment group (n=6) had grown to 1000 mm³ on average.

FIG. 7B presents results of measurement of tumor growth rate in each of the treatment and control groups for this experiment. As indicated with respect to FIG. 7A, on the tenth day following injection, tumors were first observed in all groups and these tumor sizes were used as V₀ for a calculation of growth rate (see Experimental Design). Tumor growth rates were similar in both groups until day 28 after injection, at which time tumors in the placebo group began to grow at a more rapid rate compared to tumors in leuprolide-treated mice.

As presented in FIGS. 7A and 7B, on day 45 after cell injection of U87-MG brain cancer cells, tumors growing in mice treated with leuprolide were smaller by 57% compared to tumors growing in placebo mice, and the tumors in mice treated with 4 cm of leuprolide had grown five times while tumors in the placebo mice had grown twelve times over the initial tumor size (V₀).

Experiment 8

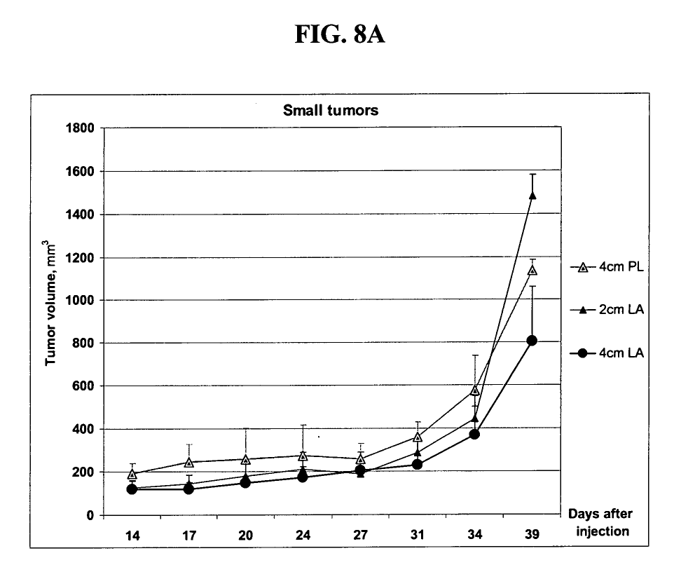

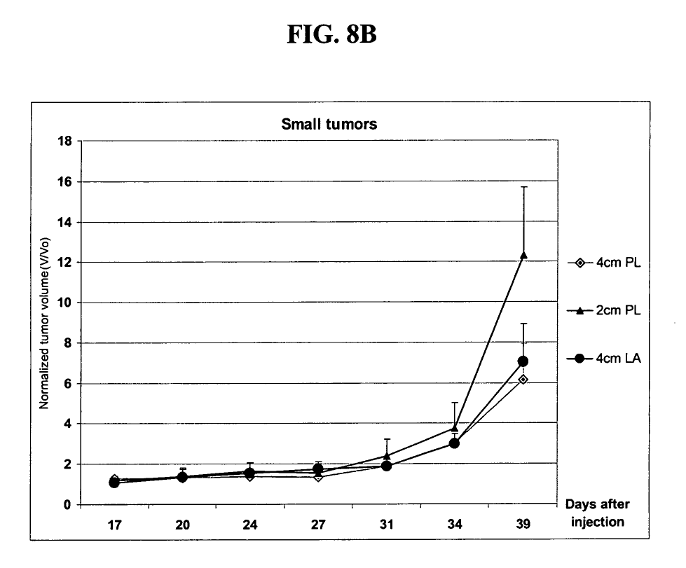

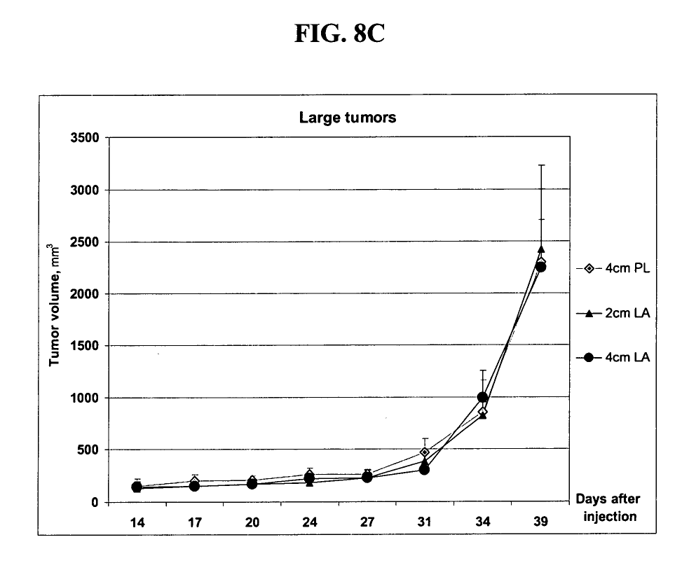

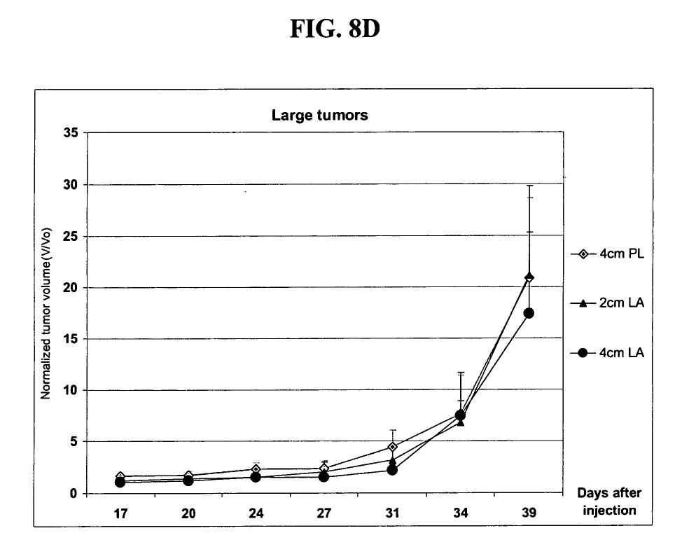

FIGS. 8A-D present results of experiments in which 5×10⁶ cells of the U87-MG human glioblastoma brain cancer cell line were injected bilaterally into three groups (two treatment groups and a control group), each with four mice. One week prior to the cell injection, a controlled-release leuprolide acetate formulation was implanted into each mouse from one of the groups. Two centimeters of leuprolide rod, providing 11.25 mg of leuprolide, or four centimeters of leuprolide rod, providing 22.5 mg of leuprolide, were implanted in each mouse of the treatment groups. Four centimeters of placebo rod (without leuprolide) was implanted one week prior to injection into each mouse of the control group.

Table 1 below presents the number of tumors formed and a subgroup analysis of small (≦4×V₀) and large (≧24×V₀) tumors. This data demonstrates that higher doses of leuprolide inhibit the formation of large tumors of the U87-MG cell line.

TABLE 1
(Experiment 8)

| | | | % of large | % of small |
|---|---|---|---|---|
| | Number | Number | tumors | tumors |
| Treatment | of mice | of tumors | (≧1600 mm³) | (≦1600 mm³) |
| 4 cm Placebo | 4 | 8 | 63 | 37 |
| 2 cm Leuprolide | 4 | 8 | 38 | 62 |
| 4 cm Leuprolide | 4 | 8 | 25 | 75 |

FIG. 8A presents results of tumor xenograft growth over time in the placebo group and the two leuprolide implant groups. As FIG. 8A shows, tumor volume measurements for small tumors were commenced on the fourteenth day following injection, when tumors were detectable in all groups. By the thirty-ninth day following injection, tumors in the control group (n=3) had grown to approximately 1150 mm³ on average, while tumors in the 2 cm treatment group (n=5) had grown to approximately 1500 mm3 on average, and tumors in the 4 cm treatment group (n=6) had grown to 800 mm³ on average.

FIG. 8B presents results of measurement of tumor growth rate in each of the treatment and control groups for this experiment. As indicated with respect to FIG. 8A, on the fourteenth day following injection, tumors were first observed in all groups and these tumor sizes were used as V₀ for a calculation of growth rate (see Experimental Design). Tumor growth rates were similar in both groups until day 34 after injection, at which time tumors in the 2 cm leuprolide group began to grow at a more rapid rate compared to tumors in the placebo and 4 cm leuprolide-treated mice.

As presented in FIGS. 8A and 8B, on day 39 after cell injection of U87-MG brain cancer cells, small tumors growing in mice treated with 4 cm leuprolide were smaller by 25 % compared to tumors growing in placebo mice; and the tumors in mice treated with 4 cm of leuprolide had grown seven times, tumors in the placebo mice had grown seven times, and tumors in mice treated with 2 cm of leuprolide had grown twelve times over the initial tumor size (V₀).

FIG. 8C presents results of tumor xenograft growth over time in the placebo group and the two leuprolide implant groups. As FIG. 8C shows, tumor volume measurements for large tumors were commenced on the fourteenth day following injection, when tumors were detectable in all groups. By the thirty-ninth day-following injection, large tumors in all groups had grown to approximately 2250 mm³.

FIG. 8D presents results of measurement of tumor growth rate for large tumors in each of the treatment and control groups for this experiment. As indicated with respect to FIG. 8C, on the fourteenth day following injection, tumors were first observed in all groups and these tumor sizes were used as V₀ for a calculation of growth rate (see Experimental Design). Tumor growth rates were similar in all groups until day 39 after injection.

As presented in Table 1 and FIGS. 8C-D, far fewer large tumors formed in mice treated with leuprolide. On day 39 after cell injection of U87-MG brain cancer cells, for the large tumors that did form, tumors growing in placebo or leuprolide-treated mice were of a similar volume and grew at similar rates.

Experiment 9

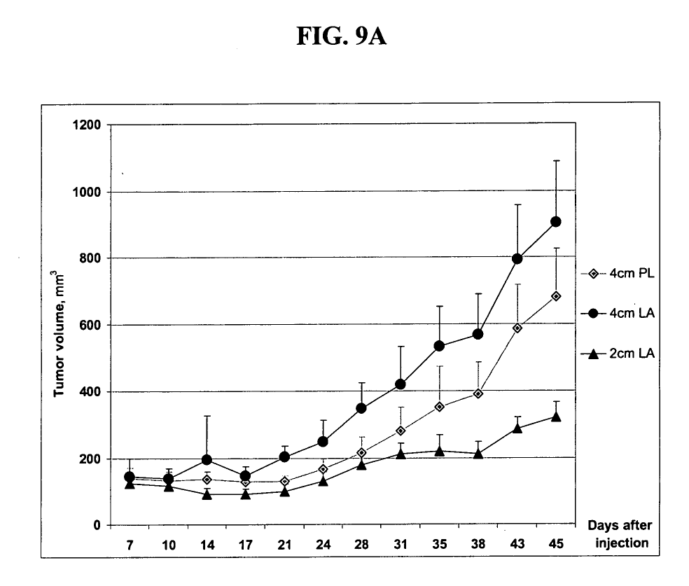

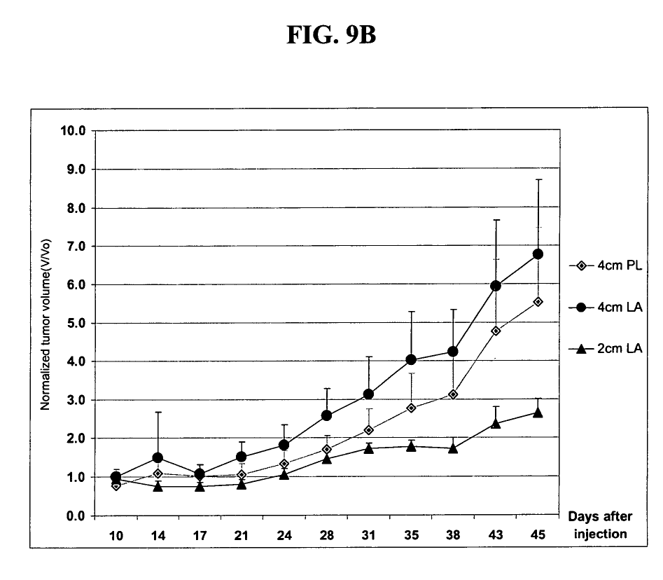

FIGS. 9A and 9B present results of experiments in which 5×10⁶ cells of the DAOY human pediatric medulloblastoma brain cancer cell line were injected bilaterally into three groups (two treatment groups and a control group), each with three mice. One week after the cell injection, a controlled-release leuprolide acetate formulation was implanted into each mouse from one of the groups. Two centimeters of leuprolide rod, providing 11.25 mg of leuprolide, or four centimeters of leuprolide rod, providing 22.5 mg of leuprolide, were implanted in each mouse of the treatment groups. Four centimeters of placebo rod (without leuprolide) were implanted one week prior to injection into each mouse of the control group.

FIG. 9A presents results of tumor xenograft growth over time in the placebo group and the two leuprolide implant groups. As FIG. 9A shows, tumor volume measurements for all tumors were commenced on the seventh day following injection, when tumors were detectable in all groups. By the forty-fifth day following injection, tumors in the control group (n=6) had grown to approximately 700 mm³ on average, while tumors in the 4 cm treatment group (n=6) had grown to approximately 900 mm³ on average, and tumors in the 2 cm leuprolide treatment group (n=6) had grown to only 300 mm³ on average.

FIG. 9B presents results of measurement of tumor growth rate in each of the treatment and control groups for this experiment. As indicated with respect to FIG. 9A, on the seventh day following injection, tumors were first observed in all groups, and these tumor sizes were used as V₀ for a calculation of growth rate (see Experimental Design). Tumor growth rates were similar in both groups until day 24 after injection, at which time tumors in the placebo and 4 cm leuprolide groups began to grow at a more rapid rate compared to tumors in the 2 cm leuprolide-treated mice.

Experiment 10

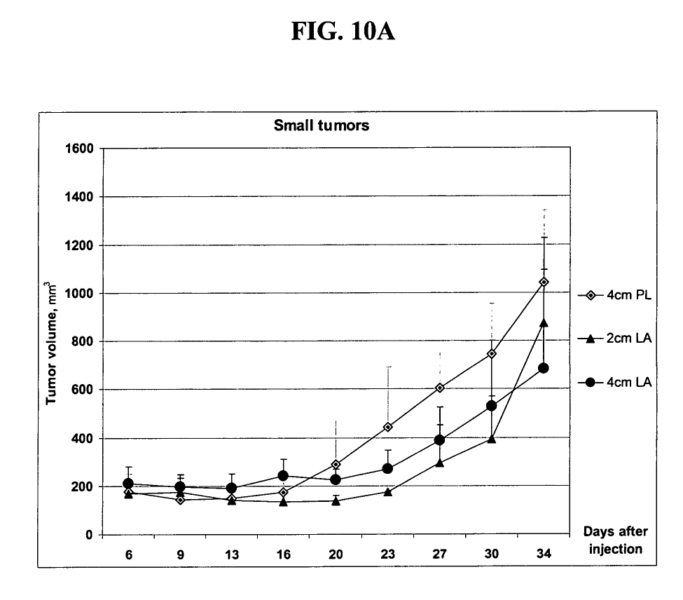

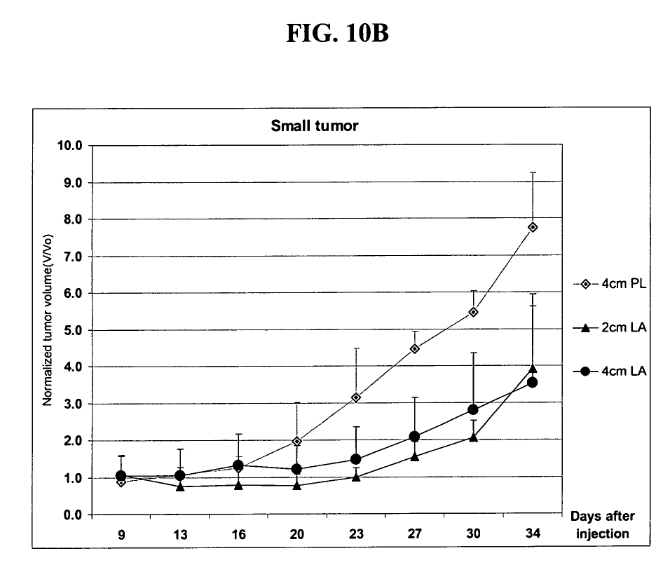

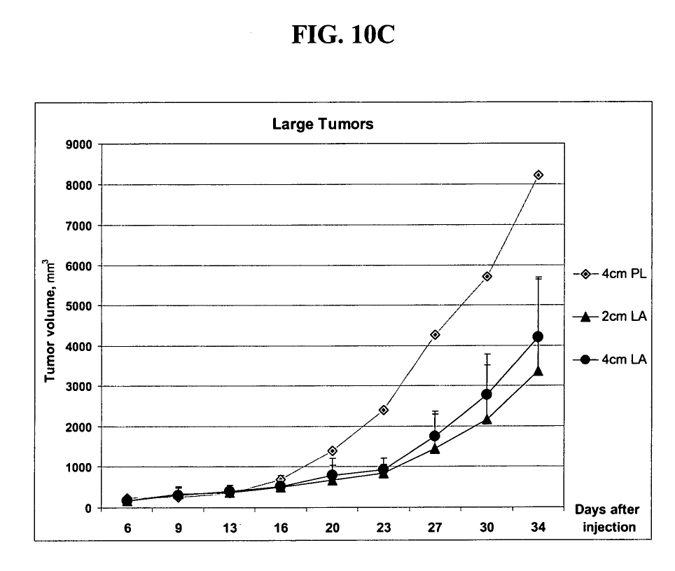

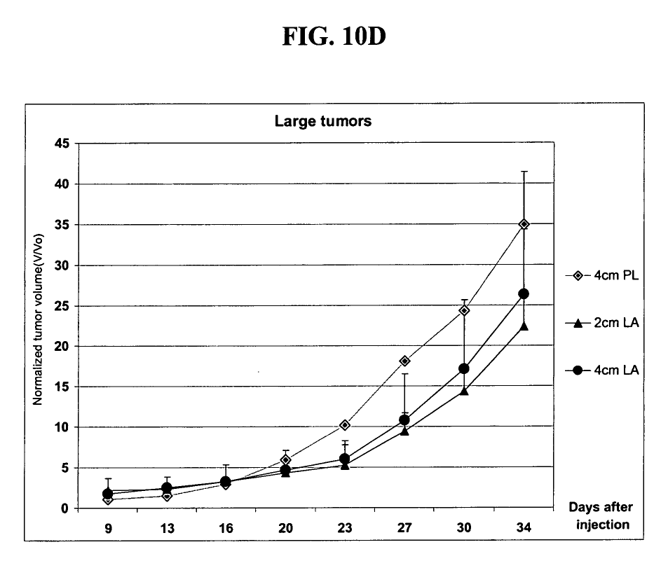

FIGS. 10A-D present results of experiments in which 5×10⁶ cells of the SK-N-MC human pediatric neuroblastoma brain cancer cell line were injected bilaterally into three groups (two treatment groups and a control group), each with three mice. One week after the cell injection, a controlled-release leuprolide acetate formulation was implanted into each mouse from one of the groups. Two centimeters of leuprolide rod, providing 11.25 mg of leuprolide, or four centimeters of leuprolide rod, providing 22.5 mg of leuprolide, were implanted in each mouse of the treatment groups. Four centimeters of placebo rod (without leuprolide) were implanted one week prior to injection into each mouse of the control group.

Table 2 below presents the number of tumors formed in three mice per group, percent of large tumors (≧10×V₀), and the average volumes of large and small tumors. While leuprolide (LA) treated mice had more large tumors, the large tumors in those mice were smaller than the large tumors in the placebo (PL) group.

TABLE 2
(Experiment 10)

| | | | | Average |
|---|---|---|---|---|
| | | % of large | Average large | small tumor |
| Treatment | # tumors | tumors | tumor V, (mm³) | V, (mm³) |
| 4 cm PL | 6 | 17 | 8215 | 1043 |
| 2 cm LA | 6 | 50 | 3361 | 874 |
| 4 cm LA | 6 | 50 | 4195 | 682 |

FIG. 10A presents results of tumor xenograft growth for small tumors over time in a placebo group and two leuprolide implant groups. As FIG. 10A shows, tumor volume measurements for small tumors were commenced on the sixth day following injection, when tumors were detectable in all groups. By the thirty-fourth day following injection, tumors in the control group (n=6) had grown to approximately 1050 mm³ on average, while tumors in the 2 cm treatment group (n=6) had grown to approximately 850 mm³ on average, and tumors in the 4 cm treatment group (n=6) had grown to 675 mm³ on average.

10B presents results of measurement of tumor growth rate for small tumors in each of the treatment and control groups for this experiment. As indicated with respect to FIG. 10A, on the sixth day following injection, tumors were first observed in all groups, and these tumor sizes were used as V₀ for a calculation of growth rate (see Experimental Design). Tumor growth rates were similar in both groups until day 16 after injection, at which time tumors in the placebo group began to grow at a more rapid rate compared to tumors in the 2 cm and 4 cm leuprolide-treated mice. Tumors in the placebo group had increased by almost eight fold while tumors in the leuprolide-treated mice had increased by only 3.5 fold.

FIG. 10C presents results of tumor xenograft growth over time in the placebo group and the two leuprolide implant groups. As FIG. 10C shows, tumor volume measurements for large tumors were commenced on the sixth day following injection, when tumors were detectable in all groups. By the thirty-fourth day following injection, large tumors in the placebo group had grown to 8200 mm³ on average, while tumors in the mice treated with 2 cm leuprolide were 3200 mm³ on average, and tumors in the mice treated with 4 cm leuprolide were 4200 mm³ on average.

FIG. 10D presents results of measurement of tumor growth rate for large tumors in each of the treatment and control groups for this experiment. As indicated with respect to FIG. 10C, on the sixth day following injection, tumors were first observed in all groups, and these tumor sizes were used as V₀ for a calculation of growth rate (see Experimental Design). Tumor growth rates were similar in both groups until day 20 after injection, at which time tumors in the placebo group began to grow at a more rapid rate compared to tumors in the 2 cm and 4 cm leuprolide-treated mice. Tumors in the placebo group had increased by almost thirty-five fold while tumors in the leuprolide-treated mice had increased by only twenty-five fold.

### Exemplary Embodiments

In embodiments of this invention, brain cancer is prevented, treated, delayed, or mitigated by administering a dosage regimen of GnRH agonists or antagonists that is at least about two to three times higher, and in other embodiments more than three times higher, than is currently approved for other indications. Since no toxic dose of GnRH agonists is believed to have been documented, other embodiments of this invention include treating, preventing, slowing the progression of, or mitigation of brain cancer by continually increasing the dose of the GnRH agonist or antagonist until a decrease in a brain cancer-specific marker is achieved, or until the patient develops adverse effects that represent greater risk or discomfort than does the risk or discomfort of the brain cancer.

In further embodiments of the invention, brain cancer would be prevented, treated, delayed, or mitigated by directly and constantly infusing GnRH agonists or antagonists into the affected tissue, for example, from a reservoir into the spinal cavity via a catheter (such as a fenestrated catheter) embedded directly into the spine of a patient. The drug is thus directly delivered to the brain through the cerebrospinal fluid rather than indirectly delivered through the bloodstream. It is well known in the art to deliver drugs by infusion through a catheter embedded directly in a part of a patient's body requiring treatment, for example, in the liver of a patient requiring chemotherapy drugs for the treatment of liver cancer.

In another embodiment of the invention, controlled release formulations of GnRH agonists or antagonists would be implanted directly into or near the brain tissue in order to prevent, treat, delay, or mitigate brain cancer, for example by implantation directly into the brain following a surgical resection of a tumor. This would allow for high brain concentrations of the GnRH agonist or antagonist while minimizing peripheral exposure.

Currently, in the course of an in vitro fertilization process, a needle may be used to inject about 1 mg/day of GnRH agonists or antagonists into a patient. According to an embodiment of the present invention, a dose of a GnRH agonist or antagonist administered for the prevention, treatment, delay, or mitigation of brain cancer, when delivered by implantation of controlled release formulations directly into or near the brain, results in serum and/or brain tissue levels up to 3 ng/ml or more. In other embodiments of the present invention, the dosage regime of GnRH agonist or antagonist to treat, prevent, mitigate, or slow the progression of brain cancer would be a physiologically equivalent dose to a dose of leuprolide in the range of 11.25 mg/month to 22.5 mg/month, or a dose of an agent resulting in daily dosages physiologically equivalent to a dose of leuprolide of approximately 0.375 mg/day to approximately 0.75 mg/day. In additional embodiments, the controlled release formulation would be formulated to maintain the tissue concentration of the GNRH agonist or antagonist at levels that produce the same or similar physiological effects as dosages of leuprolide of 7.5 mg/month, 11.25 mg/month, 22.5 mg/month, or more. In embodiments of the invention, the higher tissue concentration would be substantially sustained at a high level instead of spiking initially and briefly to a very high level and then dropping substantially.

In other embodiments of the invention, implanted controlled release formulations of GnRH agonists or antagonists would achieve a release profile that provides a substantially stable serum concentration of GnRH agonists or antagonists that is at least about two to five times the serum concentration provided by currently-known cancer treatments using GnRH agonists or antagonists (for example, treatments for prostate cancer), in which the serum concentration is substantially sustained at the higher level instead of spiking initially and briefly to a very high level and then dropping substantially. For example, an implanted controlled release formulation of the present invention for preventing, treating, delaying, or mitigating brain cancer would provide a GnRH agonist or antagonist serum concentration of at least about 3 ng/ml, in embodiments up to 10 ng/ml or more over the lifetime of the formulation. Such formulations, using polymeric controlled release technology, are available from Durect Corporation, Cupertino, Calif.

Other known methods of delivery are also suitable for administering GnRH agonists or antagonists according to the present invention, such as intramuscular injection of microspheres.

Examples of GnRH agonists or antagonists include but are not limited to Antide® brand of iturelix; Lupron® brand of leuprolide acetate; Zoladex® brand of goserelin acetate; Synarel® brand of nafarelin acetate; Trelstar Depot brand of triptorelin; Supprelin brand of histrelin; Suprefact brand of buserelin; Cetrotide® brand of cetrorelix; Plenaxis® brand of abarelix; Antagon brand of ganirelix; and degarelix (FE200486).

Embodiments of the present invention also include treating, mitigating, slowing the progression of, or preventing brain cancer by co-administering a GnRH agonist or antagonist with standard chemotherapeutic treatment.

Embodiments of the present invention further include treating, mitigating, slowing the progression of, or preventing brain cancer by co-administering a GnRH agonist or antagonist with standard radiation therapy or high-dose radiation delivered by a “gamma knife.”

Embodiments of the present invention also include treating, mitigating, slowing the progression of, or preventing brain cancer by administering a GnRH agonist or antagonist prior to surgical resection of a brain tumor.

Embodiments of the present invention additionally include treating, mitigating, slowing the progression of, or preventing brain cancer by administering a GnRH agonist or antagonist during the immediate period after a surgical resection and indefinitely thereafter to prevent tumor recurrence.

Embodiments of the present invention also include treating, mitigating, slowing the progression of, or preventing brain cancer by co-administering a GnRH agonist or antagonist with LH receptor blockers or analogues thereof, which include but are not limited to interleukin-I and anti-LH receptor immunoglobulins; co-administering a GnRH agonist or antagonist with activin receptor blockers or analogues thereof; and administering other agents, including agents not yet known, that decrease the degradation of, increase the half-life of, or increase brain tissue levels of GnRH agonists or antagonists.

Additionally, the present invention encompasses pharmaceutical formulations containing GNRH agonists and/or GnRH antagonists and which are configured to be implanted in or near brain tissue and to provide serum concentrations or certain tissue concentrations of the GnRH agonists and/or GnRH antagonists substantially higher than serum levels resulting from conventional cancer treatments using GnRH agonists or antagonists, such as, for example, conventional prostate cancer treatments. The pharmaceutical formulations could be used, for example, to treat, delay, mitigate, or prevent brain cancer.

While various embodiments of the present invention have been described throughout this specification, it should be understood that they have been presented by way of example only, and not by way of limitation. For example, the present invention is not limited to the agents illustrated or described. As such, the breadth and scope of the present invention should not be limited to any of the above-described exemplary embodiments, but should be defined in accordance with the appended claims and their equivalents.

What is claimed is:

1. A method for treating brain cancer in a patient having brain cancer, for preventing brain cancer in a patient at risk of contracting brain cancer, for decreasing the level of a brain cancer-specific marker in a patient, or for preventing or slowing the proliferation of cells of brain origin in a patient, comprising:
administering to the patient a therapeutically effective amount of at least one physiological agent that decreases or regulates blood or tissue levels, expression, production, function, or activity of at least one of luteinizing hormone (LH), LH receptors, follicle stimulating hormone (FSH), FSH receptors, an androgenic steroid, androgenic steroid receptors, an activin, and activin receptors.

2. A method for treating brain cancer in a patient having brain cancer, for preventing brain cancer in a patient at risk of contracting brain cancer, for decreasing the level of a brain cancer-specific marker in a patient, or for preventing or slowing proliferation of cells of brain origin in a patient, comprising:
administering to the patient a therapeutically effective amount of at least one physiological agent that increases or regulates blood or tissue levels, expression, production, function, or activity of at least one of gonadotropin releasing hormone (GnRH), an inhibin, beta-glycan, and a follistatin.

3. A method of preventing or inhibiting an upregulation of the cell cycle in brain-derived cells in a patient, comprising:
administering to the patient an amount of at least one physiological agent selected from the group consisting of GnRH agonists and GnRH antagonists, effective to reduce local tissue production of hormones of the hypothalamic-pituitary-gonadal (HPG) axis.

4. A method of treating brain cancer in a patient having brain cancer, comprising:
administering to the patient an amount of at least one physiological agent selected from the group consisting of GnRH agonists and GnRH antagonists, effective to achieve a blood serum level of at least about 3 ng/ml of the physiological agent for a predetermined time interval.

5. A method for treating brain cancer in a patient having brain cancer, comprising:
administering to the patient an initial dose of a GnRH agonist or a GnRH antagonist; and
monitoring for decreases in a brain cancer-specific marker level in the patient, and subsequently administering to the patient increasing doses of the GnRH agonist or the GnRH antagonist until no further decrease in a brain cancer-specific marker level in the patient is observed.

6. A method for treating brain cancer in a patient having brain cancer, comprising:
administering to the patient a therapeutically effective amount at least one physiological agent selected from the group consisting of GnRH agonists and GnRH antagonists by substantially continuously infusing the physiological agent directly into the brain of the patient so that brain cancer cells are exposed to concentrations of the physiological agent that would result from blood serum concentrations of the physiological agent of at least about 3 ng/ml.

7. The method of claim 1, wherein the at least one physiological agent is one of gonadotropin releasing hormone (GnRH), a GnRH agonist, a GnRH antagonist, an inhibin, beta-glycan, and a follistatin.

8. The method of any one of claims 1-3, wherein the at least one physiological agent is leuprolide, and the therapeutically effective amount is in the range of approximately 11.25 mg/month to at least approximately 22.5 mg/month.

9. The method of any one of claims 1-3, wherein the therapeutically effective amount of the at least one physiological agent is an amount of the physiological agent, administered or released over a predetermined time period, targeted to achieve substantially equivalent physiological effects as those resulting from a blood serum level of leuprolide of at least about 3 ng/ml of leuprolide over a period of about a month.

10. A method for treating brain cancer in a patient having brain cancer, comprising:
administering to the patient a therapeutically effective amount of at least one physiological agent selected from the group consisting of GnRH agonists and GnRH antagonists, by implanting a pharmaceutical controlled release formulation of the at least one physiological agent directly into or near the brain tissue of the patient.

11. The method of claim 10, wherein the pharmaceutical controlled release formulation is formulated to provide a serum concentration of the at least one physiological agent of at least about 3 ng/ml maintained for a period of at least about one month.

12. The method of claim 10, wherein the pharmaceutical controlled release formulation is formulated to expose brain cancer cells of the patient to concentrations of the at least one physiological agent resulting from a blood serum concentration of the at least one physiological agent of at least about 3 ng/ml for a period of at least about one month.

13. A method for treating brain cancer in a patient having brain cancer, comprising:
administering to the patient a first physiological agent selected from the group consisting of GnRH agonists and GnRH antagonists in a therapeutically effective combination with a second physiological agent selected from the group consisting of androgen synthesis blockers, analogues of androgen synthesis blockers, FSH receptor blockers, analogues of FSH receptor blockers, testosterone, testosterone analogues, LH receptor blockers, analogues of LH receptor blockers, activin blockers, and analogues of activin blockers.

14. A method for treating brain cancer in a patient having brain cancer, comprising:
administering to the patient a physiological agent that decreases the degradation of GnRH agonists or GnRH antagonists within the patient, increases the half-life of GnRH agonists or GnRH antagonists within the patient, or increases brain tissue levels of GnRH agonists or GnRH antagonists within the patient.
